(19) United States
(12) Patent Application Publication
(10) Pub. No.: US 2025/0292902 A1
(43) Pub. Date: Sep. 18, 2025

(54) LONG-TERM ANALYSIS OF HAND SWING MOVEMENT FOR ILLNESS DETECTION

(71) Applicant: Oura Health Oy, Oulu (FI)

(72) Inventors: Mika Erkkilä, Oulu (FI); Avi Halpern, Escondido, CA (US); Marko Hiltunen, Oulu (FI); Mari Pauliina Karsikas, Oulu (FI); Heli Tuulia Koskimäki, Oulu (FI)

(21) Appl. No.: 18/607,078

(22) Filed: Mar. 15, 2024

(51) Int. Cl. G16H 50/20; G06F 9/451

(52) U.S. Cl. CPC G16H 50/20; G06F 9/451

(57) ABSTRACT

Methods, systems, and devices for predicting illness using motion data are described. A wearable device may acquire physiological data from a user using one or more sensors, and the physiological data may include motion data associated with one or more arms of the user. Baseline motion data corresponding to a first time interval and additional motion data corresponding to a second time interval collected by the wearable device may be input into one or more machine learning models trained to predict illness onset or recovery based at least in part on a plurality of features associated with movement of the one or more arms of the user. The one or more machine learning models may generate an illness prediction metric based on the additional motion data and the baseline motion data, corresponding to a relative likelihood of the user experiencing one or more illnesses.

### FIELD OF TECHNOLOGY

The following relates to wearable devices and data processing, including techniques for long-term analysis of hand swing movements for illness detection.

### BACKGROUND

Some wearable devices may be configured to collect data from users associated with users, including temperature data, heart rate data, and the like. Many users have a desire for more insight regarding their physical health.

### BRIEF DESCRIPTION OF THE DRAWINGS

FIG. 1 illustrates an example of a system that supports techniques for long-term analysis of hand swing movements for illness detection in accordance with aspects of the present disclosure.

FIG. 2 illustrates an example of a system that supports techniques for long-term analysis of hand swing movements for illness detection in accordance with aspects of the present disclosure.

FIG. 3 shows an example of a system that supports techniques for long-term analysis of hand swing movements for illness detection in accordance with aspects of the present disclosure.

FIG. 4 shows an example of a flowchart that supports techniques for long-term analysis of hand swing movements for illness detection in accordance with aspects of the present disclosure.

FIG. 5 shows an example of a graphical user interface (GUI) that supports techniques for long-term analysis of hand swing movements for illness detection in accordance with aspects of the present disclosure.

FIG. 6 shows a block diagram of an apparatus that supports techniques for long-term analysis of hand swing movements for illness detection in accordance with aspects of the present disclosure.

FIG. 7 shows a block diagram of a wearable application that supports techniques for long-term analysis of hand swing movements for illness detection in accordance with aspects of the present disclosure.

FIG. 8 shows a diagram of a system including a device that supports techniques for long-term analysis of hand swing movements for illness detection in accordance with aspects of the present disclosure.

FIG. 9 shows a flowchart illustrating methods that support techniques for long-term analysis of hand swing movements for illness detection in accordance with aspects of the present disclosure.

### DETAILED DESCRIPTION

In some cases, early detection for some illnesses may be difficult to perform. For example, users with illnesses such as depression or Parkinson's Disease may experience limited symptoms until the illness has progressed. In some cases, long-term changes in a person's gait may be an early sign of some illnesses, such as reduced arm swing in at least one arm. However, measuring these changes may be difficult in a traditional doctor visit setting. For example, a single measurement of a person's arm swings during a single doctor's visit may not provide useful information to perform an early diagnosis of illnesses, and early diagnosis may require multiple measurements over time. Further, the person's gait may be affected by equipment used to obtain the measurement, and it may be difficult to ascertain whether changes in the person's arm swings are due to a developing illness or due to the method of measurement during a doctor's visit.

In accordance with examples as described herein, wearable devices may be used to perform long-term analysis of a user's gait and hand swing movements to predict onset and recovery of illness, such as Parkinson's Disease and depression. For example, by collecting gait and arm swing data over long durations of time (e.g., months, years, etc.), as opposed to collecting such data during a single doctor's visit, techniques described herein may be used to identify long-term changes in the user's gait and arm swing movements, which may be indicative of illness onset and/or recovery. In particular, by evaluating the user's gait and arm swing movements over long periods of time, the user's gait and arm swing movements may be compared to the user's own baseline data collected in the past, which may provide much better insights with predicting illness onset, and identifying illness recovery. In some examples, sensors of the wearable device used for movement (e.g., step) tracking may already be capable of collecting data associated with arm movements, and these sensors may then also be used for early diagnosis of some illnesses. Further, the wearable device is capable of collecting the arm swing data over long periods of time, which may be stored at a server or a device of the user. As such, changes in the data over time may be analyzed and used to alert the user of a potential risk in developing an illness, such as depression, chronic stress, or Parkinson's Disease. In some examples, the user may be indicated to consult a doctor for further diagnosis based on the arm swing data.

Aspects of the disclosure are initially described in the context of systems supporting physiological data collection from users via wearable devices. Aspects of the disclosure are additionally described with respect to a graphical user interface (GUI) that may be displayed on a user device or wearable device. Aspects of the disclosure are further illustrated by and described with reference to apparatus diagrams, system diagrams, and flowcharts that relate to long-term analysis of hand swing movements for illness detection.

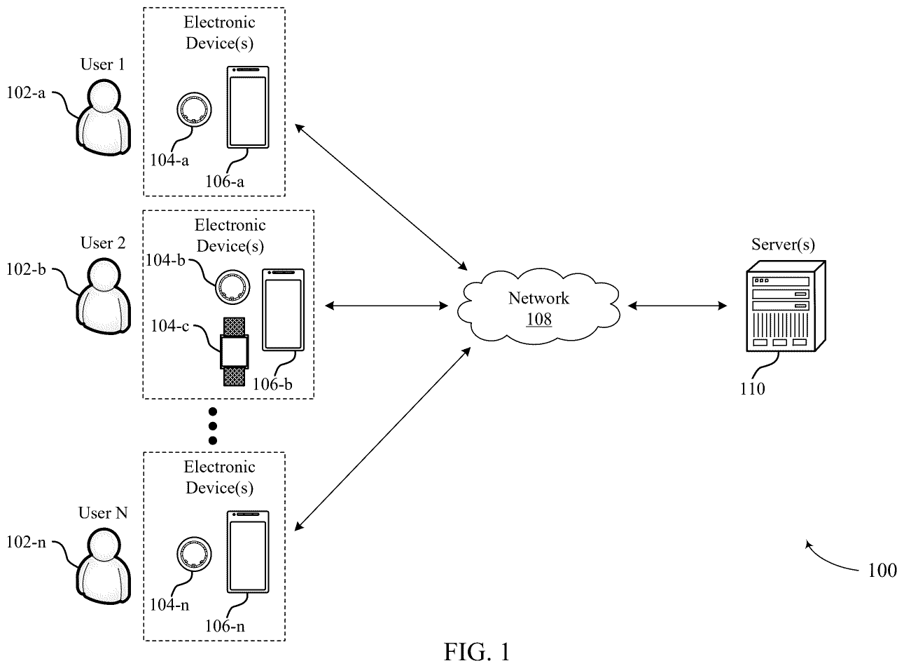

FIG. 1 illustrates an example of a system 100 that supports techniques for long-term analysis of hand swing movements for illness detection in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for performing long-term analysis of a user's gait and hand swing movements using wearable devices 104 to predict illness onset, and/or identify illness recovery. By using a wearable device 104, such as a ring wearable device 104 or a watch wearable device 104, arm swing movement may be collected without interfering with movements of a user 102, as the wearable devices 104 may be relatively light and inobtrusive. Further, as a user 102 may wear a wearable device 104 over long durations of time (e.g., months, years, etc.), techniques described herein may be used to identify long-term changes in the user's gait and arm swing movements, which may be useful for predicting illness onset and/or identifying illness recovery. In some examples, sensors of wearable devices 104 used for movement (e.g., step) tracking may already be capable of collecting data associated with arm movements, and these sensors may then also be used for early diagnosis of some illnesses, including Parkinson's Disease or depression. The gait and hand swing data collected by the wearable device 104 may be stored on a server 110 or locally at a user device 106, which may allow for analysis of the data over time (e.g., by the network 108, the server 110, the wearable device 104, or the user device 106). As such, the user 102 may be alerted of a potential risk in developing an illness, such as depression or Parkinson's Disease, based on the gait and hand swing data.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

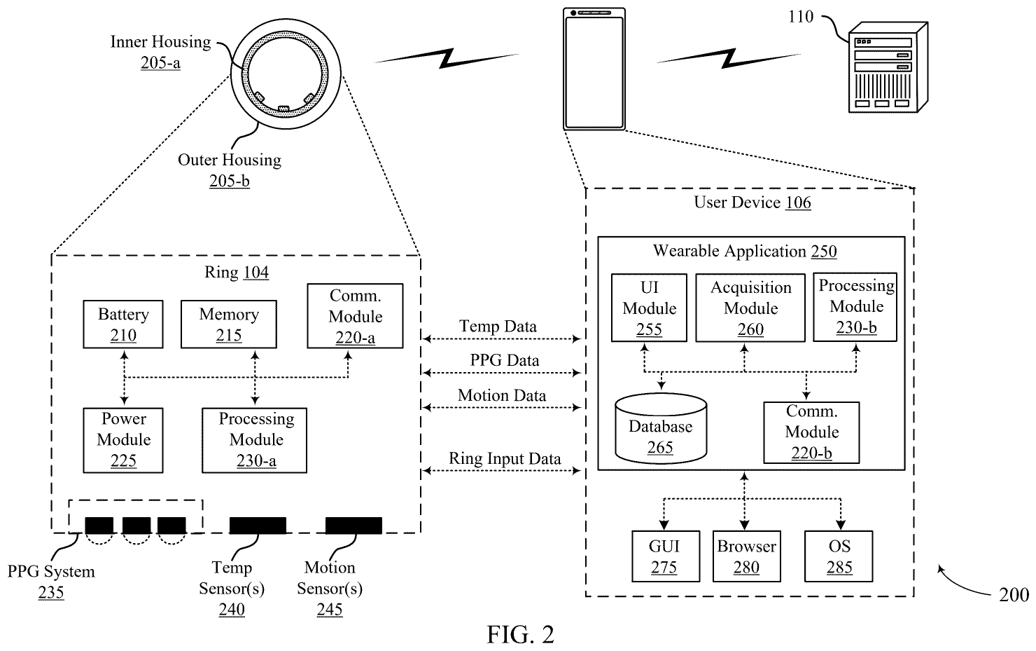

FIG. 2 illustrates an example of a system 200 that supports techniques for long-term analysis of hand swing movements for illness detection in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for performing long-term analysis of a user's gait and hand swing movements using wearable devices 104 to predict illness onset and/or identify illness recovery. A user 102 may wear a wearable device 104 over long durations of time (e.g., months, years, etc.), and the wearable device 104 may identify long-term changes in the user's gait and arm swing movements, which may be useful for predicting illness onset and/or identifying illness recovery. Further, a wearable device 104, such as a ring wearable device 104 or a watch wearable device 104, may be relatively unobtrusive. As such, arm swing movement may be collected without interfering with movements of a user 102, which may make the arm swing movement data much more useful for predicting illness onset/recovery. In some examples, the motion sensors 245 of wearable devices 104 may be used to collect arm swing movement data over time, which may be used for early diagnosis of some illnesses, including Parkinson's Disease or depression, and/or recovery from such illnesses.

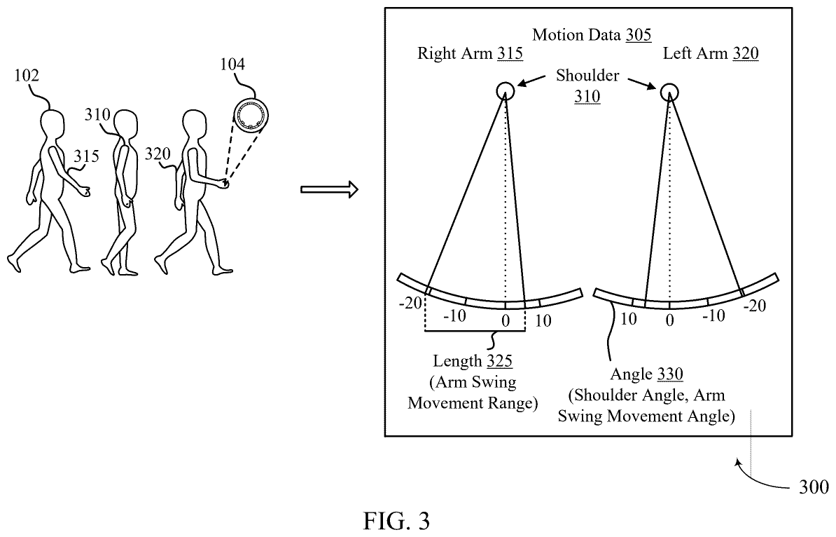

For example, the motion sensors 245 may be used to identify an angle associated with an arm swing (e.g., relative to a shoulder) while walking, a length of an arm movement while walking, or both. The data output by the motion sensors 245 (e.g., including a three-dimensional activity sensor, an accelerometer, a gyroscope, or a combination thereof) may be used to determine the shoulder/arm angle or arm swing length. Additionally, or alternatively, the motion sensors 245 may detect acceleration forces (e.g., g-forces) to measure the angle or length, which may be used for illness prediction, as described in more detail with reference to FIG. 3. In some cases, the gait and arm swing data collected by the motion sensors 245 may be stored on a server 110 or locally at a user device 106, which may allow for analysis of the data over time (e.g., by the server 110, the wearable device 104, or the user device 106). As such, the user 102 may be alerted of a potential risk in developing an illness, such as depression or Parkinson's Disease, based on the gait and hand swing data.

FIG. 3 shows an example of a system 300 that supports techniques for long-term analysis of hand swing movements for illness detection in accordance with aspects of the present disclosure. Aspects of the system 300 may implement, or be implemented by, aspects of the system 100, the system 200, or both. The system 300 illustrates the use of a wearable device 104 to collect physiological data, including motion data 305, from a user 102 for predicting illness, as described herein.

One or more wearable devices 104 may be worn at an arm of the user 102 to collect motion data 305. For example, the user may wear a ring wearable device 104 at a finger of a right arm 315, a finger of the left arm 320, or both. Additionally, or alternatively, the user 102 may wear another wearable device 104, such as a watch wearable device 104 on the right arm 315, the left arm 320, or both. In cases where the user 102 wears only one wearable device 104, the user 102 may be instructed (e.g., via a user device 106) to switch the wearable device 104 from the right arm 315 to the left arm 320, or vice-versa (e.g., alternating days or weeks). For example, the user 102 may be instructed to switch the wearable device 104 to the other arm with some threshold frequency. As such, motion data 305 may be collected at the right arm 315, the left arm 320, or both.

The motion data 305 collected by the wearable device(s) 104 may include data associated with an arm swing of the user 102. For example, the one or more wearable devices 104 may include motion sensors (e.g., accelerometers, gyroscopes, or a combination thereof) that may collect data associated with the arm swing of the user 102 while the user 102 is walking. As such, long-term changes in the gait of the user 102 may be identified, such as reduced arm swing, which may be an early sign of some illnesses, including Parkinson's Disease, depression, or other illnesses. Similarly, changes in the user's gait or arm swing may be indicative of the user 102 experiencing stress. Further, long-term analysis of the user's gait and arm swing movements may also be used to identify, quantify, or otherwise evaluate recovery from such illnesses.

In some examples, the motion data 305 may include one or more angles 330 characterizing an arm swing of at least one arm of the user 102 relative to a respective shoulder 310 of the user 102 while the user 102 is walking (e.g., shoulder angle, arm swing angle range). For example, the motion data 305 may include an angle 330 corresponding to the motion of at least one arm of the user 102 during an arm swing while the user 102 is walking. In the example illustrated in FIG. 3, the angle 330 may be around 26 degrees for the right arm 315. Additionally, or alternatively, the motion data 305 collected by the wearable devices 104 may include multiple angles 330 for each arm. For instance, the motion data 305 may include a first angle 330 measuring the forward degrees from vertical that an arm of the user 102 moves while walking, and a second angle 330 measuring backward degrees from vertical that the arm of the user 102 moves. In the example illustrated in FIG. 3, the first angle may be about five degrees for the right arm 315, while the second angle may be about 21 degrees for the right arm 315.

The motion data 305 may additionally, or alternatively, include a length 325 corresponding to the hand movement (e.g., of the right arm 315, the left arm 320, or both) of the user 102 while the user 102 is walking. That is, the length 325 may be associated with a hand swing movement range/length of the user's arms. For example, the wearable device 104 may measure the length 325 corresponding to a trajectory traveled by the wearable device 104 (e.g., located at an arm of the user 102) during an arm swing, and the wearable device 104 may measure the length 325 as horizontal distance or as an arc length.

In some examples, the motion data 305 may detect small movements (e.g., vibrations, frequencies) in the right arm 315, the left arm 320, or both. For instance, the motion data 305 may include vibrations which may correspond to tremors of the right arm 315, the left arm 320, or both. In some examples, the tremors may be detected while the user 102 is walking or at rest using the motion sensors, by detecting one or more vibrations.

In some examples, the motion data 305 acquired throughout a first time interval may be used to determine baseline motion data associated with the right arm 315, the left arm 320, or both. For example, the wearable device 104 (e.g., or a user device 106, or a server 110) may compile the motion data 305 acquired throughout the first time interval and generate the baseline motion data. In some examples, the baseline motion data 305 may correspond to an average of the motion data 305 throughout the first time period, such as an average angle 330, an average length 325, or both, for at least one arm of the user 102.

In some cases, the baseline motion data may include motion data 305 corresponding to arm swing data collected while the user 102 is walking, and the baseline motion data may exclude arm swing data collected while the user is running or performing other actions, such as exercise. For example, the wearable device 104 may determine that the user 102 is walking using the motion sensors or other sensors (e.g., using PPG data such as heart rate). In some cases, the user 102 may tag activities (e.g., time periods) with an exercise tag (e.g., or another activity tag) within an application of the user device 106, which may also indicate that the motion data 305 collected during these activities may correspond to abnormal gait, and the corresponding motion data 305 may be excluded from the baseline motion data. In some examples, the baseline motion data may be continuously updated as new data is collected from the user 102, or updated with some frequency (e.g., each day, each week).

Additionally, or alternatively, the baseline motion data may be weighted, which may omit or reduce the impact of outliers in the motion data 305 on the baseline motion data. For example, the outliers may correspond to times when the user 102 did not have a normal gait. If the user 102 is holding an object, such as a phone or dog leash for instance, the motion data 305 may not accurately represent arm swing data. As such, the wearable device 104, a user device 106, or a server 110 may determine that the user 102 is holding an object (e.g., using the motion data 305 or other data, such as PPG data), or based on the outliers, and may disregard the data or reduce the weight of the data on the baseline motion data.

The wearable device 104 may acquire additional motion data 305 throughout a second time interval subsequent to the first time interval corresponding to the baseline motion data. The second time interval may be a recent interval and may be shorter than the first time interval. For example, the second time interval may correspond to one or more previous days or weeks, while the first time interval may correspond to a longer time period for which motion data 305 has been collected for the user 102, which may be in the order of weeks, months, or years, for example. In some cases, the second time interval may at least partially overlap the first time interval, as at least some of the motion data 305 collected throughout the second time interval may be a part of the baseline motion data. In a similar manner as to the baseline motion data, the additional motion data 305 may exclude some of the motion data collected, such as if the motion data 305 was collected during exercise (e.g., as indicated by an exercise tag of the user 102, or as determined using physiological data) or while the user 102 has abnormal gait (e.g., is holding an object).

The baseline motion data acquired throughout the first time interval and the additional motion data 305 acquired throughout the second time interval may be used to predict illness onset and/or identify illness recovery, for example, by comparing the changes in the additional motion data 305 to the baseline motion data. For example, the additional motion data 305 and the baseline motion data may be input (e.g., by the wearable device 104, a user device 106, or a server 110) into one or more machine learning models (e.g., machine learning classifiers) which may be trained to predict illness onset or recovery based on features of an arm movement of at least one arm of the user 102. In other words, the one or more machine learning models may be trained to predict illness onset or recovery based on changes in the length 325 (e.g., hand swing movement range) and/or angle 330 (e.g., shoulder angle) of the user's arm swing movements over time. In some cases, the training data may include data from healthy users, data from users experiencing an illness, from users that transitioned from healthy to experiencing an illness, or a combination thereof.

In some examples, the additional movement data 305 or the baseline motion data may include an indication that the user 102 does not have a normal gait, such as if the user 102 is holding an object or engaged in one or more physical activities, for a time duration. In some cases, the user 102 being engaged in physical activity or holding an object may be determined based on the motion data 305 or based on PPG data collected by the wearable device 104 (e.g., heart rate data). As such, when the motion data 305 is input into one or more machine learning models, the one or more machine learning models may account for data collected during the time duration, for example by disregarding the data for at least one arm corresponding to the time duration for which the user 102 did not have a normal gait (e.g., when the user was exercising or holding a dog leash, for example). Accordingly, the one or more machine learning models may avoid incorrectly predicting that the user 102 is experiencing or will experience the one or more illnesses based on the user 102 having an abnormal gait for some period of time.

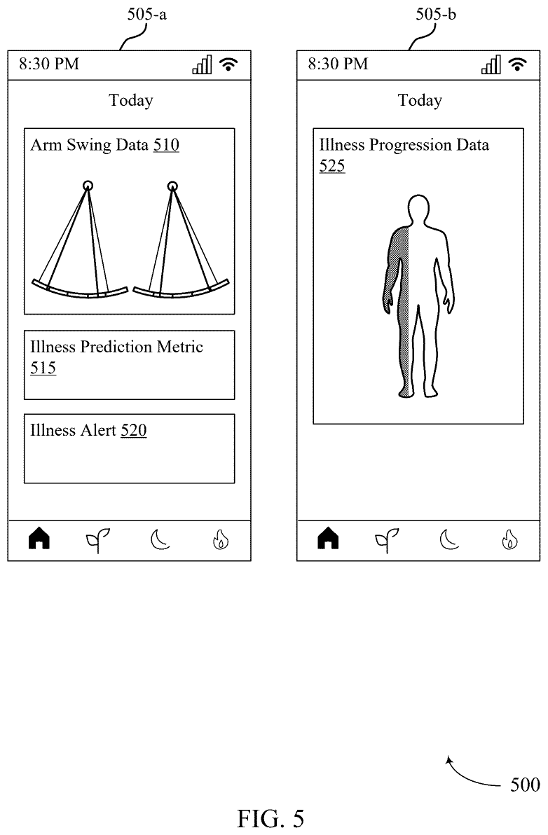

In some examples, an illness prediction metric may be generated based on inputting the baseline motion data and the additional motion data 305 to the one or more machine learning models. The illness prediction metric may correspond to a relative likelihood that the user 102 is experiencing the one or more illnesses (e.g., Parkinson's Disease, depression, or another illness), will experience the one or more illnesses, or is recovering from the one or more illnesses. In some examples, an instruction may be transmitted (e.g., by the wearable device 104, the user device 106, or a server 110) to cause a GUI of the wearable device 104 (e.g., a watch wearable device 104) or a user device 106 to display information related to the illness prediction metric. Displaying information associated with the illness prediction metric via a GUI is described in more detail with reference to FIG. 5.

In some examples, one or more illnesses, such as Parkinson's Disease and depression, may be associated with a decrease in arm swing while walking. For example, a user 102 may experience a decrease in the angle 330 or the length 325 over long periods of time, which may be indicative of the one or more illnesses and may be used to predict illness onset. As such, the illness prediction metric may be based on the angle 330 (e.g., shoulder angle range) corresponding to at least one arm of the user 102 decreasing during the additional motion data 305 relative to the baseline motion data. Additionally, or alternatively, the illness prediction metric may be based on the length 325 (e.g., hand swing movement range) corresponding to at least one arm of the user 102 experiencing a decrease during the additional motion data 305 relative to the baseline motion data. In some cases, the illness prediction metric may be based on the decrease in angle 330, the length 325, or both, of at least one arm of the user 102 decreasing more than a threshold amount relative to the baseline motion data.

Conversely, the machine learning model may be configured to identify that the user is recovering from illness based on increases in the angle 330 and/or length 325 of the arm(s) of the user 102 over time. For example, the user 102 may experience a decrease in the angle 330, the length 325, or both, of at least one arm from an illness (e.g., depression, Parkinson's Disease, or another illness), condition, or medical procedure (e.g., surgery). In some cases, the baseline motion data may correspond to data collected while the user 102 experiences the decrease in the angle 330 or the length 325 due to the illness, condition, or medical procedure. Additionally, or alternatively, the user 102 may experience limping, which may be detected using the data collected by the wearable device 104. The one or more machine learning models may identify an increase in the angle 330, the length 325, or both, for one or more arms of the user 102 (e.g., relative to the baseline motion data), which be associated with a relative likelihood that the user 102 may be recovering from the illness, condition (e.g., limping), or medical procedure. In some cases, the illness prediction metric may be based on the increase in the angle 330, the length 325, or both, of at least one arm of the user 102 increasing more than a threshold amount relative to the baseline motion data, which may be indicative of recovery.

In some cases, the one or more machine learning models may be used to identify one or more motion frequencies for respective time intervals/durations of the additional motion data 305. In particular, the machine learning models may be configured to classify motion identified within respective time durations as being attributable to the user's normal gait, or due to tremors. For instance, the additional motion data 305 may include one or more tremors that may be experienced by the user 102. The one or more machine learning models may identify a respective frequency associated with a respective tremor, which may be used to determine illness. For example, some illnesses, such as Parkinson's Disease, may be associated with tremors having vibration frequencies within a threshold range, such as between 4 to 6 Hz. As such, the one or more machine learning models may classify one or more time intervals as tremors based on having a vibration frequency within the threshold range. In some examples, one or more motion frequencies may be outside the threshold range. As such, the one or more machine learning models may classify the corresponding one or more time intervals as corresponding to a gait of the user, as these time intervals may correspond to frequencies caused by motion of the user 102 while walking or running. The relative likelihood of the user 102 experiencing an illness may be affected based on the quantity of tremors identified having a vibration frequency within the threshold range, for example.

In some examples, one or more motion frequencies detected by the wearable device 104 may exceed a threshold value. For instance, the frequency may be or appear larger than a frequency that the wearable device 104 is able to detect. As such, the wearable device 104 may receive (e.g., from a user device 106, a server 110) an instruction to increase a measurement sampling frequency of one or more sensors of the wearable device 104. As such, the wearable device 104 may collect motion data 305 using the increased measurement sampling frequency, which may enable the wearable device 104 to detect finer frequencies or tremors.

In some examples, it may be beneficial to detect frequencies while the user 102 is at rest (e.g., in a relaxed state), as vibrations corresponding to tremors may be masked by the movement of the arm of the user 102 while the user 102 is walking or running. As such, when the user device 106, a server 110, or the wearable device 104 determine that the user is at rest, the measurement sampling frequency may be increased to enhance tremor detection by the wearable device 104. In some cases, the wearable device 104 may receive an instruction to increase the measurement sampling frequency from the server 110 or the user device 106. In some examples, the user device 106, the server 110, or the wearable device 104 may determine that the user 102 is at rest based on the motion data 305, or other data, such as PPG data (e.g., heart rate).

In some cases, a user 102 experiencing a decrease in arm movement (e.g., angle 330 or length 325) in one arm, but not the other arm, may indicate that the user 102 may experience or be experiencing the one or more illnesses. Moreover, differences in the length 325 and/or angle 330 on different sides of the body may be used to determine which side of the brain/body is more or less affected by illness. As such, the one or more machine learning models may determine, based on the motion data 305, a first symmetry metric corresponding to a relative symmetry of the hand swing movement (e.g., the respective lengths 325) range between the right arm 315 and the left arm 320. Additionally, or alternatively, the one or more machine learning models may determine a second symmetry metric corresponding to a relative symmetry of the shoulder angle range (e.g., the respective angles 330) between the right arm 315 and the left arm 320. The one or more machine learning metrics may also determine a third symmetry metric associated with a movement symmetry between the right arm 315 and the left arm 320.

A change in the first, second, or third symmetry metrics in the additional motion data relative to the baseline motion data may be indicative of illness or illness offset. For instance, if the one or more symmetry metrics have changed more than a threshold value relative to the baseline motion data (i.e. decrease in symmetry metric indicating larger difference between length 325 and/or angle 330 on the user's left and right sides), the one or more symmetry metrics may indicate that the user 102 may have a higher relative likelihood of experiencing the one or more illnesses. Additionally, or alternatively, if one or more of the symmetry metrics are below a threshold value, which may be associated with a baseline symmetry value corresponding to the one or more illnesses, the one or more symmetry metrics may indicate that the user 102 may have a higher relative likelihood of experiencing the one or more illnesses. As such, the illness prediction metric may be based on the one or more symmetry metrics or on changes within the one or more symmetry metrics relative to the baseline motion data.

In some cases, the additional motion data 305 may be used to identify one or more physical activities that the user 102 may be engaged in. For example, the one or more machine learning models may compare the additional motion data 305 to reference motion data (e.g., training data) associated with one or more physical activities (e.g., sports, exercises, activities such as running, etc.). If additional motion data 305 collected from the user 102 matches the reference motion data (e.g., with a threshold similarity of arm swing data, for example), the one or more machine learning models may determine that the user 102 engaged in the corresponding physical activity during a corresponding time period. In some examples, the additional motion data 305 may be used to classify a workout of the user 102 (e.g., initiated by the user 102, such as via the user device 106) or create a tag for the workout or for a movement. The tag may be displayed to the user 102 via a GUI of the user device 106 (e.g., or the wearable device 104), which may aid the user 102 in classifying and keeping track of workouts and activities.

Accordingly, by acquiring motion data 305 from the user 102 via one or more wearable devices 104, including arm swing data, motion frequency data, and symmetry data, an illness prediction metric may be generated, which may help the user 102 be aware of potential health risks and identify a relative likelihood of experiencing one or more illnesses.

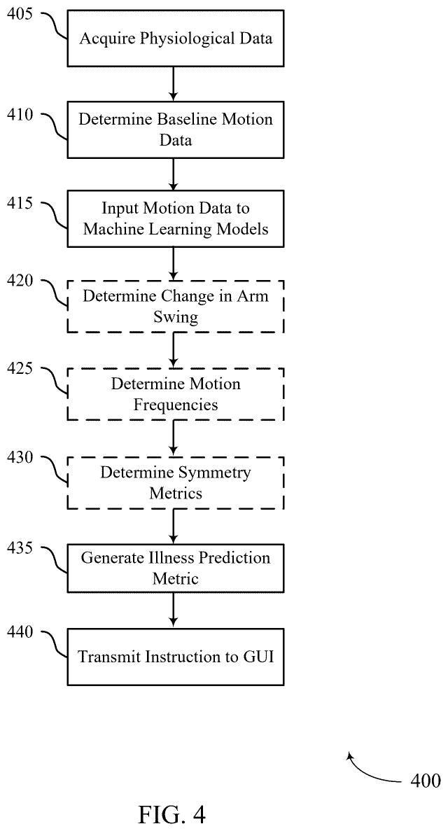

FIG. 4 shows an example of a flowchart 400 that supports techniques for long-term analysis of hand swing movements for illness detection in accordance with aspects of the present disclosure. Aspects of the flowchart 400 may implement, or be implemented by, aspects of the system 100, the system 200, the system 300, or any combination thereof. In some examples, the steps of the flowchart 400 may be performed in a different order than shown. Additionally, or alternatively, some steps may be added to the flowchart 400, and some steps may be omitted or optional. The flowchart 400 may illustrate steps for predicting one or more illnesses, such as Parkinson's Disease or depression, based on motion data collected by a wearable device 104.

At 405, the wearable device 104, which may be worn on an arm of a user 102, may acquire physiological data including motion data (e.g., motion data 305). The motion data may include one or more angles corresponding to an arm swing of the user 102 relative to a shoulder of the user 102, a length corresponding to the hand movement of the user 102 while walking, or both. The motion data 305 may also include small movements in at least one arm of the user, such as vibrations which may correspond to tremors.

At 410, the wearable device 104, a server 110, a user device 106, or another device may determine baseline motion data associated with at least one arm of the user using the motion data acquired throughout a first time interval. In some examples, the baseline motion data may correspond to an average of the motion data throughout the first time period, such as an average angle, an average length, or both, for at least one arm of the user 102. In some cases, the baseline motion data may be continuously updated as new data is collected from the user 102.

At 415, the baseline motion data acquired throughout the first time interval and additional motion data acquired throughout a second time interval may be input into one or more machine learning models. The one or more machine learning models may be trained to predict illness onset or recovery based on features associated with movement of the one or more arms of the user within the additional motion data relative to the baseline motion data. For example, the features may include a first feature associated with a change in a hand swing movement range of the one or more arms, a second feature associated with a change in a shoulder angle range of the one or more arms, or both. In some examples, the one or more machine learning models may be trained using training data, which may include data (e.g., motion data, hand swing movement range data, shoulder angle range data) from healthy users, from users experiencing from an illness, from users that transitioned from healthy to experiencing an illness, or a combination thereof.

At 420, the one or more machine learning models may identify a change in hand swing movement range, shoulder angle range, or both, for at least one arm of the user. For example, the one or more machine learning models may identify a change in hand swing movement range, shoulder angle range, or both, in the additional motion data relative to the baseline motion data. In some cases, identifying the change in the hand swing movement range, the shoulder angle range, or both, may be based on the change (e.g., a decrease) exceeding a threshold value.

At 425, the one or more machine learning models may identify one or more motion frequencies associated with one or more time intervals within the second time interval corresponding to the additional motion data. The one or more machine learning models may classify a time interval as being associated with a tremor based on the respective motion frequency identified within the time interval. Additionally, or alternatively, the one or more machine learning models may classify a time interval as being associated with a gait of the user (e.g., other movement of the user), based on the respective motion corresponding to the time interval.

At 430, the one or more machine learning models may determine a first symmetry metric corresponding to a relative symmetry of the hand swing movement between a first arm of the user 102 and a second arm of the user 102. Additionally, or alternatively, the one or more machine learning models may determine a second symmetry metric corresponding to a relative symmetry of the shoulder angle range between the first arm and the second arm. The one or more machine learning models may also determine a third symmetry metric associated with a movement symmetry between first arm and the second arm. In some examples, the one or more machine learning models may identify a change in the first symmetry metric, the second symmetry metric, the third symmetry metric, or a combination thereof, based on the additional motion data relative to the baseline motion data.

At 435, the one or more machine learning models may generate an illness prediction metric corresponding to a relative likelihood of the user 102 experiencing the one or more illnesses (e.g., Parkinson's Disease, depression, chronic or short-term stress, or another illness), and/or recovering from the one or more illnesses. The illness prediction metric may be based on the changes in the hand swing movement range, shoulder angle range, or both, within the additional motion data relative to the baseline motion data. For example, a decrease in the hand swing movement range, shoulder angle range, or both, may be indicative of a higher relative likelihood of experiencing the one or more illnesses. Conversely, an increase in the hand swing movement range, shoulder angle range, or both, may be indicative that the user is recovering from one or more illnesses.

Additionally, or alternatively, the illness prediction metric may be based on the identified one or more motion frequencies. For example, deviations of the user's arm swing movements relative to the user's baseline arm swing movements within the second time interval may be associated with a higher relative likelihood of experiencing the one or more illnesses. Additionally, or alternatively, the illness prediction metric may be based on the first symmetry metric, the second symmetric metric, the third symmetry metric, or a combination thereof. For example, a change in the symmetry metrics observed within the additional motion data relative to the baseline motion data may indicate illness progression at a part of the body of the user 102, or may be associated with a higher relative likelihood of experiencing the one or more illnesses.

At 440, an instruction may be transmitted by the wearable device 104, the user device 106, or the server 110, to cause a GUI of the wearable device 104 or the user device 106 to display information related to the illness prediction metric. For example, the GUI may display the illness prediction metric as a numerical value corresponding to the relative likelihood of experiencing the one or more illnesses. Additionally, or alternatively, the GUI may display an alert for the user to seek a medical diagnosis based on the illness prediction metric exceeding a threshold value. In some examples, the GUI may display an indication of a part of the body of the user 102 that may be affected by the one or more illnesses, for example, based on the symmetry metrics.

FIG. 5 shows an example of a GUI 500 that supports techniques for long-term analysis of hand swing movements for illness detection in accordance with aspects of the present disclosure. Aspects of the GUI 500 may implement, or be implemented by, aspects of the system 100, the system 200, the system 300, the flowchart 400, or any combination thereof. The GUI 500 may be displayed on a screen of a device of a user 102, such as a user device 106, or a wearable device 104, such as a wrist-worn wearable device 104 (e.g., a watch) that may include a screen. The GUI 500 illustrates a series of application pages 505 which may be displayed to the user via the GUI 500, such as the application page 505-a and the application page 505-b. In some examples, the information illustrated within each application page 505 may be displayed in different order than shown or may be displayed in a different application page 505. Additionally, or alternatively, some information shown may be omitted, while other information not shown may also be included.

In some examples, one or more instructions may be transmitted by the wearable device 104, the user device 106, or a server 110 to cause the GUI 500 to display information related to physiological and motion data collected from the user 102 via the wearable device 104. For example, the one or more instructions may cause the GUI 500 to display arm swing data 510. The arm swing data 510 may illustrate an indication of an arm swing for one or both arms of the user 102 (e.g., if the user 102 wears a single wearable device 104 on one arm, arm swing data 510 for the one arm may be shown). In some examples, the arm swing data 510 may illustrate hand swing movement range, shoulder angle range, or both, as described herein, for at least one arm of the user 102. In some examples, the arm swing data 510 may correspond to motion data collected during a recent time interval, such as the past day, week, or month. In some cases, the arm swing data 510 may additionally, or alternatively, illustrate baseline arm swing data 510, which may correspond to motion data collected during a longer time interval (e.g., weeks, months, years). For example, the baseline arm swing data 510 may be illustrated as thinner lines, which may allow the user 102 to identify changes in the arm swing data 510 relative to the baseline.

In some examples, information related to the illness prediction metric 515 for the user 102 may be displayed by the GUI 500 based on the one or more instructions. For example, the illness prediction metric 515 may be displayed as a numerical value, such as a percentage representing a relative likelihood of the user 102 experiencing one or more illnesses (e.g., Parkinson's Disease, depression, stress). Additionally, or alternatively, the illness prediction metric 515 may be displayed as (e.g., or as part of) a general health score (e.g., a wellness score) corresponding to an overall health evaluation for the user 102 (e.g., which may be based on the motion data), or as an indication of a stress level of the user.

In some cases, if the illness prediction metric 515 indicates a relatively high likelihood that the user 102 is experiencing or will experience the one or more illnesses (e.g., if the illness prediction metric satisfies one or more thresholds), the one or more instructions may cause the GUI 500 to display an illness alert 520. The illness alert 520 may suggest (e.g., instruct) the user 102 to schedule an appointment with their doctor, seek medical attention regarding the one or more illnesses, seek a medical diagnosis for the one or more illnesses, etc., Additionally, or alternatively, the illness alert 520 may display one or more suggestions (e.g., instructions) for the user 102 to treat the one or more illnesses or treat symptoms associated with the one or more illnesses (e.g., to reduce stress, or to treat depression symptoms).

Additionally, or alternatively, the illness prediction metric 515 may be associated with a relative likelihood that the user 102 is recovering from the one or more illnesses, from a condition (e.g., limping), or from a procedure (e.g., surgery). The GUI 500 may display (e.g., as part of the illness alert 520, the illness prediction metric 515, the arm swing data 510) one or more insights associated with the recovery of the user 102. For instance, the GUI 500 may illustrate (e . . . g, using one or more graphs) an change (e.g., an increase) of an arm swing movement over time, which may be associated with the recovery of the user 102. In some examples, the change may be displayed from a time associated with onset of the one or more illnesses, the condition, or the procedure (e.g., the surgery), which may be input by the user 102 (e.g., via the GUI 500). As such, the GUI 500 may illustrate how the user 102 is recovering from illnesses, conditions, or procedures.

In some examples, one or more machine learning models may identify a progression of the one or more illnesses (e.g., illness progression data 525) relative to a lateral plane of the body of the user 102 based on the collected motion data. For instance, as shown in the illness progression data 525 in FIG. 5, the one or more machine learning models may identify that a left side of the user is affected by the illness, for example, based on a quantity of tremors identified in the motion data, or based on a change in arm movement range identified in the motion data (e.g., the arm swing data 510). In some cases, for example, the motion data may indicate a decrease in arm swing movement for one arm of the user 102. Additionally, or alternatively, illness progression relative to the lateral plane of the body (as illustrated via the illness progression data 525) may be identified using one or more symmetry metrics determined by the one or more machine learning models, as described herein with reference to FIG. 3. The GUI 500 may display the illness progression data 525 of the illness relative to the lateral plane, which may indicate to the user 102 which portion of their body may be affected by the one or more illnesses. Additionally, or alternatively, the illness progression data 525 may show how values of arm swing movements/ranges change over time in one or more charts, timelines, etc.

Accordingly, one or more wearable devices 104 may be used to collect motion data from a user, which may be used in predicting the onset or progression of one or more illnesses. The user 102 may be informed via the GUI 500, which may also instruct the user 102 to perform actions that may benefit the health of the user 102, such as seeking a medical diagnosis.

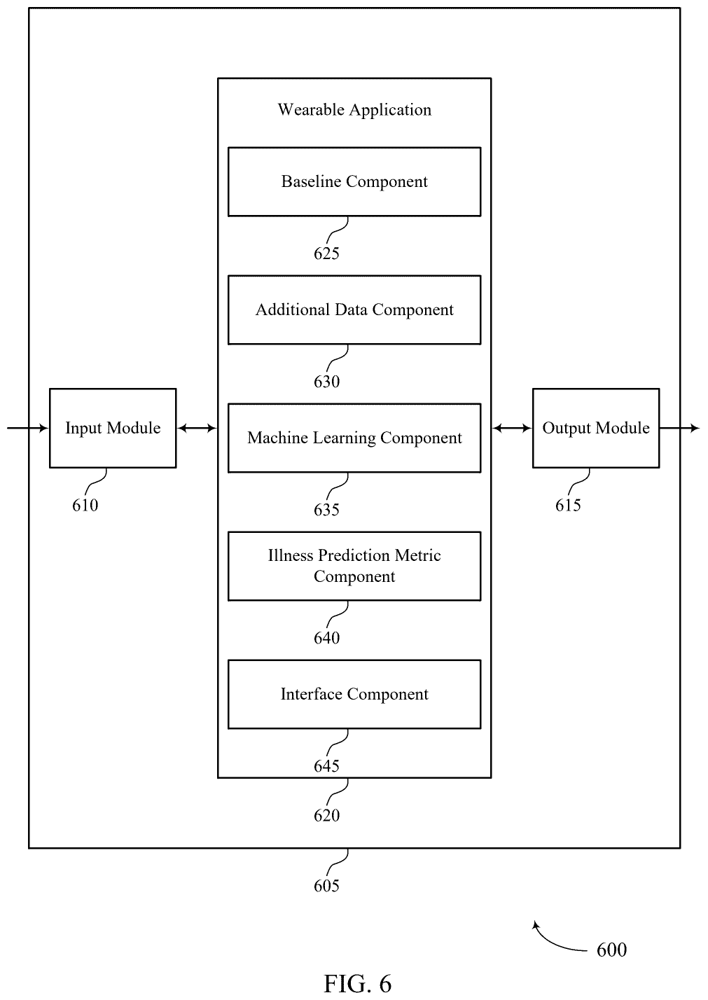

FIG. 6 shows a block diagram 600 of a device 605 that supports long-term analysis of hand swing movement for illness detection in accordance with aspects of the present disclosure. The device 605 may include an input module 610, an output module 615, and a wearable application 620. The device 605, or one or more components of the device 605 (e.g., the input module 610, the output module 615, the wearable application 620), may include at least one processor, which may be coupled with at least one memory, to support the described techniques. Each of these components may be in communication with one another (e.g., via one or more buses).

The input module 610 may provide a means for receiving information such as packets, user data, control information, or any combination thereof associated with various information channels (e.g., control channels, data channels, information channels related to illness detection techniques). Information may be passed on to other components of the device 605. The input module 610 may utilize a single antenna or a set of multiple antennas.

The output module 615 may provide a means for transmitting signals generated by other components of the device 605. For example, the output module 615 may transmit information such as packets, user data, control information, or any combination thereof associated with various information channels (e.g., control channels, data channels, information channels related to illness detection techniques). In some examples, the output module 615 may be co-located with the input module 610 in a transceiver module. The output module 615 may utilize a single antenna or a set of multiple antennas.

For example, the wearable application 620 may include a baseline component 625, an additional data component 630, a machine learning component 635, an illness prediction metric component 640, an interface component 645, or any combination thereof. In some examples, the wearable application 620, or various components thereof, may be configured to perform various operations (e.g., receiving, monitoring, transmitting) using or otherwise in cooperation with the input module 610, the output module 615, or both. For example, the wearable application 620 may receive information from the input module 610, send information to the output module 615, or be integrated in combination with the input module 610, the output module 615, or both to receive information, transmit information, or perform various other operations as described herein.

The wearable application 620 may support predicting illness onset in accordance with examples as disclosed herein. The baseline component 625 may be configured as or otherwise support a means for acquiring, using one or more sensors of a wearable device, baseline motion data associated with one or more arms of a user, the baseline motion data acquired throughout a first time interval. The additional data component 630 may be configured as or otherwise support a means for acquiring, using the one or more sensors of the wearable device, additional motion data associated with the one or more arms of the user, the additional motion data acquired via the one or more sensors of the wearable device throughout a second time interval that is subsequent to the first time interval. The machine learning component 635 may be configured as or otherwise support a means for inputting the baseline motion data and the additional motion data into one or more machine learning models, the one or more machine learning models trained to predict illness onset based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both. The illness prediction metric component 640 may be configured as or otherwise support a means for generating, using the one or more machine learning models, an illness prediction metric based at least in part on the plurality of features within the additional motion data, the illness prediction metric associated with a relative likelihood that the user is experiencing or will experience one or more illnesses. The interface component 645 may be configured as or otherwise support a means for transmitting, the wearable device, a user device, or both, an instruction configured to cause a GUI to display information associated with the illness prediction metric.

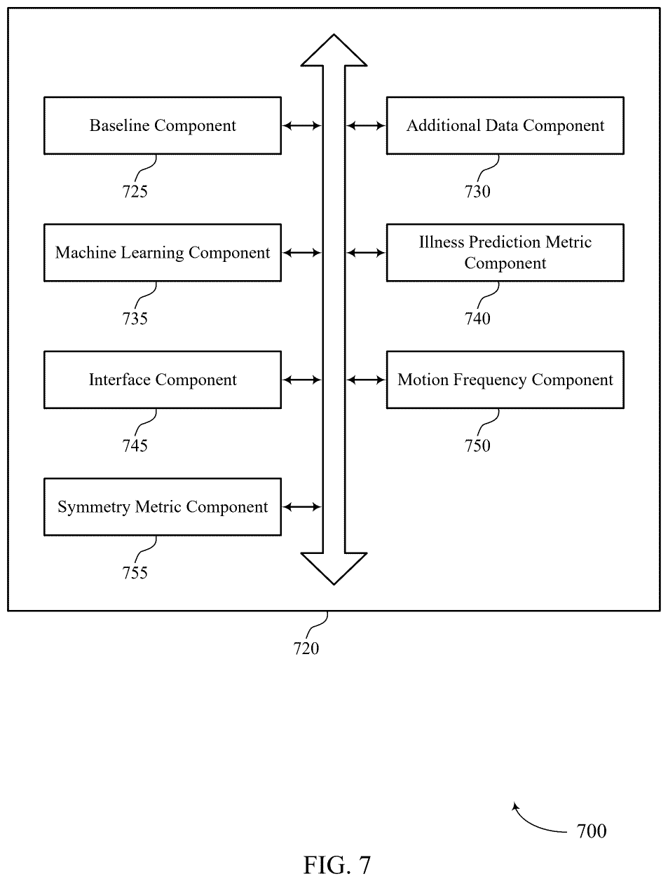

FIG. 7 shows a block diagram 700 of a wearable application 720 that supports long-term analysis of hand swing movement for illness detection in accordance with aspects of the present disclosure. The wearable application 720 may be an example of aspects of a wearable application or a wearable application 620, or both, as described herein. The wearable application 720, or various components thereof, may be an example of means for performing various aspects of long-term analysis of hand swing movement for illness detection as described herein. For example, the wearable application 720 may include a baseline component 725, an additional data component 730, a machine learning component 735, an illness prediction metric component 740, an interface component 745, a motion frequency component 750, a symmetry metric component 755, or any combination thereof. Each of these components, or components of subcomponents thereof (e.g., one or more processors, one or more memories), may communicate, directly or indirectly, with one another (e.g., via one or more buses).

The wearable application 720 may support predicting illness onset in accordance with examples as disclosed herein. The baseline component 725 may be configured as or otherwise support a means for acquiring, using one or more sensors of a wearable device, baseline motion data associated with one or more arms of a user, the baseline motion data acquired throughout a first time interval. The additional data component 730 may be configured as or otherwise support a means for acquiring, using the one or more sensors of the wearable device, additional motion data associated with the one or more arms of the user, the additional motion data acquired via the one or more sensors of the wearable device throughout a second time interval that is subsequent to the first time interval. The machine learning component 735 may be configured as or otherwise support a means for inputting the baseline motion data and the additional motion data into one or more machine learning models, the one or more machine learning models trained to predict illness onset based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both. The illness prediction metric component 740 may be configured as or otherwise support a means for generating, using the one or more machine learning models, an illness prediction metric based at least in part on the plurality of features within the additional motion data, the illness prediction metric associated with a relative likelihood that the user is experiencing or will experience one or more illnesses. The interface component 745 may be configured as or otherwise support a means for transmitting, the wearable device, a user device, or both, an instruction configured to cause a GUI to display information associated with the illness prediction metric.

In some examples, the motion frequency component 750 may be configured as or otherwise support a means for identifying, using the one or more machine learning models, one or more motion frequencies associated with one or more time intervals of the additional motion data associated with the one or more arms of the user. In some examples, the machine learning component 735 may be configured as or otherwise support a means for classifying, using the one or more machine learning models, the one or more time intervals of the additional motion data as being associated with a gait of the user or a tremor based at least in part on the one or more motion frequencies, wherein the plurality of features of the additional motion data further comprise the one or more motion frequencies associated with the one or more arms of the user.

In some examples, the one or more time intervals are classified based at least in part on comparing the one or more motion frequencies with one or more frequency thresholds associated with the one or more illnesses.

In some examples, the motion frequency component 750 may be configured as or otherwise support a means for identifying that at least a first portion of the additional motion data is associated with a motion frequency that exceeds a threshold motion frequency. In some examples, the motion frequency component 750 may be configured as or otherwise support a means for transmitting an instruction to the wearable device based at least in part on the motion frequency exceeding the threshold motion frequency, wherein the instruction is configured to cause the wearable device to increase a measurement sampling frequency of the one or more sensors, wherein at least a second portion of the additional motion data is acquired by the one or more sensors in accordance with the increased measurement sampling frequency, and wherein the illness prediction metric is generated based at least in part on transmitting the instruction to increase the measurement sampling frequency.

In some examples, the one or more arms of the user comprise a first arm and a second arm, and the symmetry metric component 755 may be configured as or otherwise support a means for determining, using the one or more machine learning models, a first symmetry metric associated with a relative symmetry of the hand swing movement range between the first arm and the second arm, a second symmetry metric associated with a relative symmetry of the shoulder angle range between the first arm and the second arm, or both, wherein generating the illness prediction metric is based at least in part on the first symmetry metric, the second symmetry metric, or both.

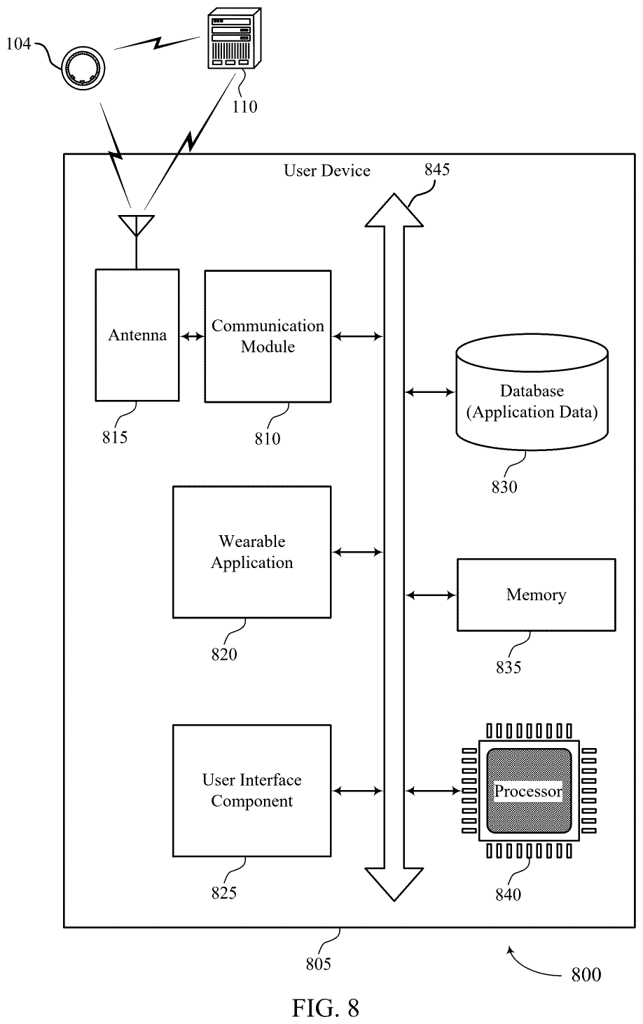

FIG. 8 shows a diagram of a system 800 including a device 805 that supports long-term analysis of hand swing movement for illness detection in accordance with aspects of the present disclosure. The device 805 may be an example of or include components of a device 405 as described herein. The device 805 may include an example of a user device 106, as described previously herein. The device 805 may include components for bi-directional communications including components for transmitting and receiving communications with a wearable device 104 and a server 110, such as a wearable application 820, a communication module 810, one or more antennas 815, a user interface component 825, a database (application data) 830, at least one memory 835, and at least one processor 840. These components may be in electronic communication or otherwise coupled (e.g., operatively, communicatively, functionally, electronically, electrically) via one or more buses (e.g., a bus 845).

The communication module 810 may manage input and output signals for the device 805 via the antenna 815. The communication module 810 may include an example of the communication module 220-b of the user device 106 shown and described in FIG. 2. In this regard, the communication module 810 may manage communications with the ring 104 and the server 110, as illustrated in FIG. 2. The communication module 810 may also manage peripherals not integrated into the device 805. In some cases, the communication module 810 may represent a physical connection or port to an external peripheral. In some cases, the communication module 810 may utilize an operating system such as iOS®, ANDROID®, MS-DOS®, MS-WINDOWS®, OS/2®, UNIX®, LINUX®, or another known operating system. In other cases, the communication module 810 may represent or interact with a wearable device (e.g., ring 104), modem, a keyboard, a mouse, a touchscreen, or a similar device. In some cases, the communication module 810 may be implemented as part of the processor 840. In some examples, a user may interact with the device 805 via the communication module 810, user interface component 825, or via hardware components controlled by the communication module 810.

In some cases, the device 805 may include a single antenna 815. However, in some other cases, the device 805 may have more than one antenna 815, which may be capable of concurrently transmitting or receiving multiple wireless transmissions. The communication module 810 may communicate bi-directionally, via the one or more antennas 815, wired, or wireless links as described herein. For example, the communication module 810 may represent a wireless transceiver and may communicate bi-directionally with another wireless transceiver. The communication module 810 may also include a modem to modulate the packets, to provide the modulated packets to one or more antennas 815 for transmission, and to demodulate packets received from the one or more antennas 815.

The user interface component 825 may manage data storage and processing in a database 830. In some cases, a user may interact with the user interface component 825. In other cases, the user interface component 825 may operate automatically without user interaction. The database 830 may be an example of a single database, a distributed database, multiple distributed databases, a data store, a data lake, or an emergency backup database.

The memory 835 may include RAM and ROM. The memory 835 may store computer-readable, computer-executable software including instructions that, when executed, cause the processor 840 to perform various functions described herein. In some cases, the memory 835 may contain, among other things, a BIOS which may control basic hardware or software operation such as the interaction with peripheral components or devices.

The processor 840 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 840 may be configured to operate a memory array using a memory controller. In other cases, a memory controller may be integrated into the processor 840. The processor 840 may be configured to execute computer-readable instructions stored in a memory 835 to perform various functions (e.g., functions or tasks supporting a method and system for sleep staging algorithms).

The wearable application 820 may support predicting illness onset in accordance with examples as disclosed herein. For example, the wearable application 820 may be configured as or otherwise support a means for acquiring, using one or more sensors of a wearable device, baseline motion data associated with one or more arms of a user, the baseline motion data acquired throughout a first time interval. The wearable application 820 may be configured as or otherwise support a means for acquiring, using the one or more sensors of the wearable device, additional motion data associated with the one or more arms of the user, the additional motion data acquired via the one or more sensors of the wearable device throughout a second time interval that is subsequent to the first time interval. The wearable application 820 may be configured as or otherwise support a means for inputting the baseline motion data and the additional motion data into one or more machine learning models, the one or more machine learning models trained to predict illness onset based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both. The wearable application 820 may be configured as or otherwise support a means for generating, using the one or more machine learning models, an illness prediction metric based at least in part on the plurality of features within the additional motion data, the illness prediction metric associated with a relative likelihood that the user is experiencing or will experience one or more illnesses. The wearable application 820 may be configured as or otherwise support a means for transmitting, the wearable device, a user device, or both, an instruction configured to cause a graphical user interface (GUI) to display information associated with the illness prediction metric.

By including or configuring the wearable application 820 in accordance with examples as described herein, the device 805 may support techniques for

The wearable application 820 may include an application (e.g., “app”), program, software, or other component which is configured to facilitate communications with a ring 104, server 110, other user devices 106, and the like. For example, the wearable application 820 may include an application executable on a user device 106 which is configured to receive data (e.g., physiological data) from a ring 104, perform processing operations on the received data, transmit and receive data with the servers 110, and cause presentation of data to a user 102.

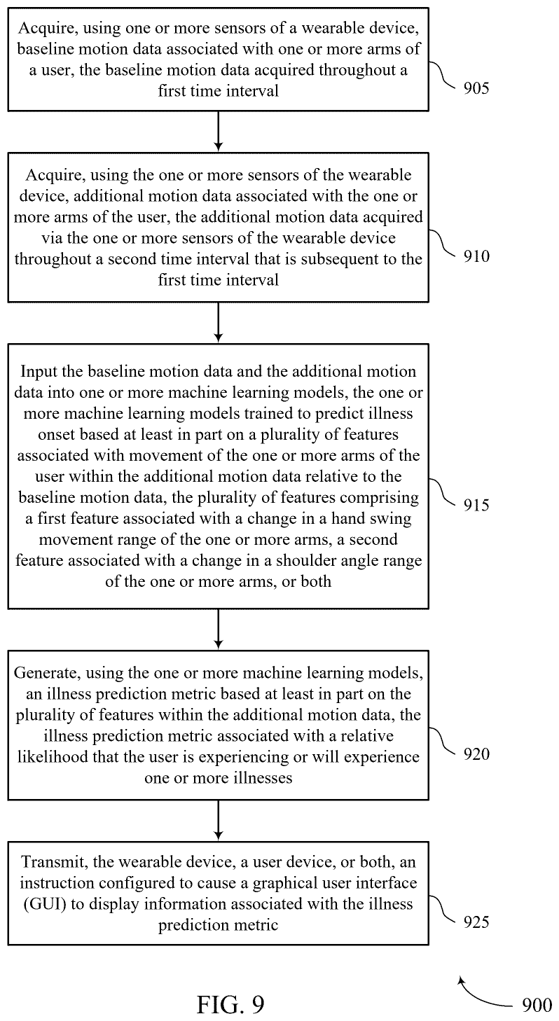

FIG. 9 shows a flowchart illustrating a method 900 that supports techniques for long-term analysis of hand swing movements for illness detection in accordance with aspects of the present disclosure. The operations of the method 900 may be implemented by a user device or its components as described herein. For example, the operations of the method 900 may be performed by a user device as described with reference to FIGS. 1 through 8. For example, the operations of the method 900 may be performed by a user device, a wearable device, or another device, as described with reference to FIGS. 1 through 8. In some examples, a device may execute a set of instructions to control the functional elements of the user device to perform the described functions. Additionally, or alternatively, the device may perform aspects of the described functions using special-purpose hardware.

At 905, the method may include acquiring, using one or more sensors of a wearable device, baseline motion data associated with one or more arms of a user, the baseline motion data acquired throughout a first time interval. The operations of 905 may be performed in accordance with examples as disclosed herein. In some examples, aspects of the operations of 905 may be performed by a wearable device 104, as described herein with reference to FIGS. 1 through 8. For example, the operations of 905 may be performed by a baseline component 725 as described with reference to FIG. 7.

At 910, the method may include acquiring, using the one or more sensors of the wearable device, additional motion data associated with the one or more arms of the user, the additional motion data acquired via the one or more sensors of the wearable device throughout a second time interval that is subsequent to the first time interval. The operations of 910 may be performed in accordance with examples as disclosed herein. In some examples, aspects of the operations of 910 may be performed by a wearable device 104, as described herein with reference to FIGS. 1 through 8. For example, the operations of 905 may be performed by an additional data component 730 as described with reference to FIG. 7.

At 915, the method may include inputting the baseline motion data and the additional motion data into one or more machine learning models, the one or more machine learning models trained to predict illness onset based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both. The operations of 915 may be performed in accordance with examples as disclosed herein. In some examples, aspects of the operations of 915 may be performed by a wearable device 104, a user device 106, a network 108, a server 110, or any combination thereof, as described herein with reference to FIGS. 1 through 8. For example, the operations of 905 may be performed by a machine learning component 735 as described with reference to FIG. 7.

At 920, the method may include generating, using the one or more machine learning models, an illness prediction metric based at least in part on the plurality of features within the additional motion data, the illness prediction metric associated with a relative likelihood that the user is experiencing or will experience one or more illnesses. The operations of 920 may be performed in accordance with examples as disclosed herein. In some examples, aspects of the operations of 920 may be performed by a wearable device 104, a user device 106, a network 108, a server 110, another device, or any combination thereof, as described herein with reference to FIGS. 1 through 8. For example, the operations of 905 may be performed by an illness prediction metric component 740 as described with reference to FIG. 7.

At 925, the method may include transmitting, to the wearable device, a user device, or both, an instruction configured to cause a GUI to display information associated with the illness prediction metric. The operations of 925 may be performed in accordance with examples as disclosed herein. In some examples, aspects of the operations of 925 may be performed by a wearable device 104, a user device 106, a network 108, a server 110, another device, or any combination thereof, as described herein with reference to FIGS. 1 through 8. For example, the operations of 905 may be performed by an interface component 745 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method for predicting illness onset by an apparatus is described. The method may include acquiring, using one or more sensors of a wearable device, baseline motion data associated with one or more arms of a user, the baseline motion data acquired throughout a first time interval, acquiring, using the one or more sensors of the wearable device, additional motion data associated with the one or more arms of the user, the additional motion data acquired via the one or more sensors of the wearable device throughout a second time interval that is subsequent to the first time interval, inputting the baseline motion data and the additional motion data into one or more machine learning models, the one or more machine learning models trained to predict illness onset based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both, generating, using the one or more machine learning models, an illness prediction metric based at least in part on the plurality of features within the additional motion data, the illness prediction metric associated with a relative likelihood that the user is experiencing or will experience one or more illnesses, and transmitting, the wearable device, a user device, or both, an instruction configured to cause a GUI to display information associated with the illness prediction metric.

An apparatus for predicting illness onset is described. The apparatus may include one or more memories storing processor executable code, and one or more processors coupled with the one or more memories. The one or more processors may individually or collectively be operable to execute the code to cause the apparatus to acquire, using one or more sensors of a wearable device, baseline motion data associated with one or more arms of a user, the baseline motion data acquired throughout a first time interval, acquire, using the one or more sensors of the wearable device, additional motion data associated with the one or more arms of the user, the additional motion data acquired via the one or more sensors of the wearable device throughout a second time interval that is subsequent to the first time interval, input the baseline motion data and the additional motion data into one or more machine learning models, the one or more machine learning models trained to predict illness onset based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both, generate, using the one or more machine learning models, an illness prediction metric based at least in part on the plurality of features within the additional motion data, the illness prediction metric associated with a relative likelihood that the user is experiencing or will experience one or more illnesses, and transmit, the wearable device, a user device, or both, an instruction configured to cause a GUI to display information associated with the illness prediction metric.

Another apparatus for predicting illness onset is described. The apparatus may include means for acquiring, using one or more sensors of a wearable device, baseline motion data associated with one or more arms of a user, the baseline motion data acquired throughout a first time interval, means for acquiring, using the one or more sensors of the wearable device, additional motion data associated with the one or more arms of the user, the additional motion data acquired via the one or more sensors of the wearable device throughout a second time interval that is subsequent to the first time interval, means for inputting the baseline motion data and the additional motion data into one or more machine learning models, the one or more machine learning models trained to predict illness onset based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both, means for generating, using the one or more machine learning models, an illness prediction metric based at least in part on the plurality of features within the additional motion data, the illness prediction metric associated with a relative likelihood that the user is experiencing or will experience one or more illnesses, and means for transmitting, the wearable device, a user device, or both, an instruction configured to cause a GUI to display information associated with the illness prediction metric.

A non-transitory computer-readable medium storing code for predicting illness onset is described. The code may include instructions executable by one or more processors to acquire, using one or more sensors of a wearable device, baseline motion data associated with one or more arms of a user, the baseline motion data acquired throughout a first time interval, acquire, using the one or more sensors of the wearable device, additional motion data associated with the one or more arms of the user, the additional motion data acquired via the one or more sensors of the wearable device throughout a second time interval that is subsequent to the first time interval, input the baseline motion data and the additional motion data into one or more machine learning models, the one or more machine learning models trained to predict illness onset based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both, generate, using the one or more machine learning models, an illness prediction metric based at least in part on the plurality of features within the additional motion data, the illness prediction metric associated with a relative likelihood that the user is experiencing or will experience one or more illnesses, and transmit, the wearable device, a user device, or both, an instruction configured to cause a GUI to display information associated with the illness prediction metric.

Some examples of the method, apparatus, and non-transitory computer-readable medium described herein may further include operations, features, means, or instructions for identifying, using the one or more machine learning models, one or more motion frequencies associated with one or more time intervals of the additional motion data associated with the one or more arms of the user and classifying, using the one or more machine learning models, the one or more time intervals of the additional motion data as being associated with a gait of the user or a tremor based at least in part on the one or more motion frequencies, wherein the plurality of features of the additional motion data further comprise the one or more motion frequencies associated with the one or more arms of the user.

In some examples of the method, apparatus, and non-transitory computer-readable medium described herein, the one or more time intervals may be classified based at least in part on comparing the one or more motion frequencies with one or more frequency thresholds associated with the one or more illnesses.

Some examples of the method, apparatus, and non-transitory computer-readable medium described herein may further include operations, features, means, or instructions for identifying that at least a first portion of the additional motion data may be associated with a motion frequency that exceeds a threshold motion frequency and transmitting an instruction to the wearable device based at least in part on the motion frequency exceeding the threshold motion frequency, wherein the instruction may be configured to cause the wearable device to increase a measurement sampling frequency of the one or more sensors, wherein at least a second portion of the additional motion data may be acquired by the one or more sensors in accordance with the increased measurement sampling frequency, and wherein the illness prediction metric may be generated based at least in part on transmitting the instruction to increase the measurement sampling frequency.

In some examples of the method, apparatus, and non-transitory computer-readable medium described herein, the one or more arms of the user comprise a first arm and a second arm and the method, apparatuses, and non-transitory computer-readable medium may include further operations, features, means, or instructions for determining, using the one or more machine learning models, a first symmetry metric associated with a relative symmetry of the hand swing movement range between the first arm and the second arm, a second symmetry metric associated with a relative symmetry of the shoulder angle range between the first arm and the second arm, or both, wherein generating the illness prediction metric may be based at least in part on the first symmetry metric, the second symmetry metric, or both.

An apparatus predicting illness onset is described. The apparatus may include a wearable device comprising one or more sensors configured to acquire physiological data from a user, the physiological data comprising at least motion data associated with one or more arms of the user, a user device communicatively coupled with the wearable device, one or more processors communicatively coupled with the wearable device and the user device, wherein the one or more processors are configured to, acquire baseline motion data associated with the one or more arms of the user, the baseline motion data acquired via the one or more sensors of the wearable device throughout a first time interval, acquire additional motion data associated with the one or more arms of the user, the additional motion data acquired via the one or more sensors of the wearable device throughout a second time interval that is subsequent to the first time interval, input the baseline motion data and the additional motion data into one or more machine learning models, the one or more machine learning models trained to predict illness onset or recovery based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both, generate, using the one or more machine learning models, an illness prediction metric based at least in part on the plurality of features within the additional motion data, the illness prediction metric associated with a relative likelihood that the user is experiencing, will experience, or is recovering from one or more illnesses, and transmit, the wearable device, a user device, or both, an instruction configured to cause a GUI to display information associated with the illness prediction metric.

In some examples of the apparatus, the one or more processors may be further configured to identify, using the one or more machine learning models, one or more motion frequencies associated with one or more time intervals of the additional motion data associated with the one or more arms of the user and classify, using the one or more machine learning models, the one or more time intervals of the additional motion data as being associated with a gait of the user or a tremor based at least in part on the one or more motion frequencies, wherein the plurality of features of the additional motion data further comprise the one or more motion frequencies associated with the one or more arms of the user.

In some examples of the apparatus, the one or more time intervals may be classified based at least in part on comparing the one or more motion frequencies with one or more frequency thresholds associated with the one or more illnesses.

In some examples of the apparatus, the one or more processors may be further configured to identify that at least a first portion of the additional motion data may be associated with a motion frequency that exceeds a threshold motion frequency and transmit an instruction to the wearable device based at least in part on the motion frequency exceeding the threshold motion frequency, wherein the instruction may be configured to cause the wearable device to increase a measurement sampling frequency of the one or more sensors, wherein at least a second portion of the additional motion data may be acquired by the one or more sensors in accordance with the increased measurement sampling frequency, and wherein the illness prediction metric may be generated based at least in part on transmitting the instruction to increase the measurement sampling frequency.

In some examples of the apparatus, the one or more processors may be further configured to identify that the user may be in a rest or relaxed state during the second time interval based at least in part on at least a first portion of the additional motion data, other physiological data acquired during the second time interval, or both and transmit an instruction to the wearable device based at least in part on the user being in the rest or relaxed state during the first time interval, wherein the instruction may be configured to cause the wearable device to increase a measurement sampling frequency of the one or more sensors, wherein at least a second portion of the additional motion data may be acquired by the one or more sensors in accordance with the increased measurement sampling frequency, and wherein the illness prediction metric may be generated based at least in part on transmitting the instruction to increase the measurement sampling frequency.

In some examples of the apparatus, the one or more processors may be further configured to determine, using the one or more machine learning models, that the change in the shoulder angle range of the one or more arms relative to the baseline motion data of the user exceeds a threshold deviation value, wherein generating the illness prediction metric may be based at least in part on the change in the shoulder angle range exceeding the threshold deviation value.

In some examples of the apparatus, the baseline motion data associated with the one or more arms of the user comprises a baseline shoulder angle range of the one or more arms and the change in the shoulder angle range may be determined relative to the baseline shoulder angle range.

In some examples of the apparatus, the one or more processors may be further configured to determine, using the one or more machine learning models, that the change in the hand swing movement range of the one or more arms relative to the baseline motion data of the user exceeds a threshold deviation value, wherein generating the illness prediction metric may be based at least in part on the change in the hand swing movement range the threshold deviation value.

In some examples of the apparatus, the baseline motion data associated with the one or more arms of the user comprises a baseline hand swing movement range of the one or more arms and the change in the hand swing movement range may be determined relative to the baseline hand swing movement range.

In some examples of the apparatus, the one or more processors may be further configured to determine, using the one or more machine learning models, a first symmetry metric associated with a relative symmetry of the hand swing movement range between the first arm and the second arm, a second symmetry metric associated with a relative symmetry of the shoulder angle range between the first arm and the second arm, or both, wherein generating the illness prediction metric may be based at least in part on the first symmetry metric, the second symmetry metric, or both.

In some examples of the apparatus, the plurality of features used to predict illness onset further comprise a third feature associated with a movement symmetry metric between the first arm and the second arm.

In some examples of the apparatus, generating the illness prediction metric comprises determine, using the one or more machine learning models, an illness progression of the one or more illnesses relative to a lateral plane of a body of the user based at least in part on the first symmetry metric, the second symmetry metric, or both, wherein the instruction may be configured to cause the GUI to display an indication of the illness progression relative to the lateral plane of the body of the user.

In some examples of the apparatus, the one or more processors may be further configured to transmit, to the wearable device, the user device, or both, an additional instruction configured to cause the GUI to display a message to instruct the user to seek a medical diagnosis based at least in part on the illness prediction metric exceeding a threshold value. In some examples of the apparatus, the one or more illnesses comprise depression, Parkinson's disease, or both.

In some examples of the apparatus, the one more processors may be further configured to determine that the user may be holding an object within a hand of the one or more arms during the second time interval based at least in part on physiological data acquired via the one or more sensors of the wearable device and input an indication that the user may be holding the object into the one or more machine learning models, wherein generating the illness prediction metric may be based at least in part on the indication.

In some examples of the apparatus, the wearable device comprises a wearable ring device configured to be worn around a finger of the one or more arms of the user. In some examples of the apparatus, the one or more sensors comprise one or more accelerometers, one or more gyroscopes, or both.

In some examples of the apparatus, the one or more processors may be further configured to determine one or more physical activities engaged in by the user during the second time interval based at least in part on the baseline motion data and the additional motion data.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A system for predicting illness onset, comprising:
a wearable device comprising one or more sensors configured to acquire physiological data from a user, the physiological data comprising at least motion data associated with one or more arms of the user;
a user device communicatively coupled with the wearable device; and
one or more processors communicatively coupled with the wearable device and the user device, wherein the one or more processors are configured to:
acquire baseline motion data associated with the one or more arms of the user, the baseline motion data acquired via the one or more sensors of the wearable device throughout a first time interval;
acquire additional motion data associated with the one or more arms of the user, the additional motion data acquired via the one or more sensors of the wearable device throughout a second time interval that is subsequent to the first time interval;
input the baseline motion data and the additional motion data into one or more machine learning models, the one or more machine learning models trained to predict illness onset or recovery based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both;
generate, using the one or more machine learning models, an illness prediction metric based at least in part on the plurality of features within the additional motion data, the illness prediction metric associated with a relative likelihood that the user is experiencing, will experience, or is recovering from one or more illnesses; and
transmit, to the wearable device, a user device, or both, an instruction configured to cause a graphical user interface (GUI) to display information associated with the illness prediction metric.

2. The system of claim 1, wherein the one or more processors are further configured to:
identify, using the one or more machine learning models, one or more motion frequencies associated with one or more time intervals of the additional motion data associated with the one or more arms of the user; and
classify, using the one or more machine learning models, the one or more time intervals of the additional motion data as being associated with a gait of the user or a tremor based at least in part on the one or more motion frequencies, wherein the plurality of features of the additional motion data further comprise the one or more motion frequencies associated with the one or more arms of the user.

3. The system of claim 2, wherein the one or more time intervals are classified based at least in part on comparing the one or more motion frequencies with one or more frequency thresholds associated with the one or more illnesses.

4. The system of claim 1, wherein the one or more processors are further configured to:
identify that at least a first portion of the additional motion data is associated with a motion frequency that exceeds a threshold motion frequency; and
transmit an instruction to the wearable device based at least in part on the motion frequency exceeding the threshold motion frequency, wherein the instruction is configured to cause the wearable device to increase a measurement sampling frequency of the one or more sensors, wherein at least a second portion of the additional motion data is acquired by the one or more sensors in accordance with the increased measurement sampling frequency, and wherein the illness prediction metric is generated based at least in part on transmitting the instruction to increase the measurement sampling frequency.

5. The system of claim 1, wherein the one or more processors are further configured to:
identify that the user is in a rest or relaxed state during the second time interval based at least in part on at least a first portion of the additional motion data, other physiological data acquired during the second time interval, or both; and
transmit an instruction to the wearable device based at least in part on the user being in the rest or relaxed state during the first time interval, wherein the instruction is configured to cause the wearable device to increase a measurement sampling frequency of the one or more sensors, wherein at least a second portion of the additional motion data is acquired by the one or more sensors in accordance with the increased measurement sampling frequency, and wherein the illness prediction metric is generated based at least in part on transmitting the instruction to increase the measurement sampling frequency.

6. The system of claim 1, wherein the one or more processors are further configured to:
determine, using the one or more machine learning models, that the change in the shoulder angle range of the one or more arms relative to the baseline motion data of the user exceeds a threshold deviation value, wherein generating the illness prediction metric is based at least in part on the change in the shoulder angle range exceeding the threshold deviation value.

7. The system of claim 6, wherein the baseline motion data associated with the one or more arms of the user comprises a baseline shoulder angle range of the one or more arms, and wherein the change in the shoulder angle range is determined relative to the baseline shoulder angle range.

8. The system of claim 1, wherein the one or more processors are further configured to:
determine, using the one or more machine learning models, that the decrechange ase in the hand swing movement range of the one or more arms relative to the baseline motion data of the user exceeds a threshold deviation value, wherein generating the illness prediction metric is based at least in part on the change in the hand swing movement range the threshold deviation value.

9. The system of claim 8, wherein the baseline motion data associated with the one or more arms of the user comprises a baseline hand swing movement range of the one or more arms, and wherein the change in the hand swing movement range is determined relative to the baseline hand swing movement range.

10. The system of claim 1, wherein the one or more arms of the user comprise a first arm and a second arm, wherein the one or more processors are further configured to:
determine, using the one or more machine learning models, a first symmetry metric associated with a relative symmetry of the hand swing movement range between the first arm and the second arm, a second symmetry metric associated with a relative symmetry of the shoulder angle range between the first arm and the second arm, or both, wherein generating the illness prediction metric is based at least in part on the first symmetry metric, the second symmetry metric, or both.

11. The system of claim 10, wherein the plurality of features used to predict illness onset further comprise a third feature associated with a movement symmetry metric between the first arm and the second arm.

12. The system of claim 10, wherein generating the illness prediction metric comprises:
determine, using the one or more machine learning models, an illness progression of the one or more illnesses relative to a lateral plane of a body of the user based at least in part on the first symmetry metric, the second symmetry metric, or both, wherein the instruction is configured to cause the GUI to display an indication of the illness progression relative to the lateral plane of the body of the user.

13. The system of claim 1, wherein the one or more processors are further configured to:
transmit, to the wearable device, the user device, or both, an additional instruction configured to cause the GUI to display a message to instruct the user to seek a medical diagnosis based at least in part on the illness prediction metric exceeding a threshold value.

14. The system of claim 1, wherein the one or more illnesses comprise depression, Parkinson's disease, or both.

15. The system of claim 1, wherein the one more processors are further configured to:
determine that the user is holding an object within a hand of the one or more arms during the second time interval based at least in part on physiological data acquired via the one or more sensors of the wearable device; and
input an indication that the user is holding the object into the one or more machine learning models, wherein generating the illness prediction metric is based at least in part on the indication.

16. The system of claim 1, wherein the wearable device comprises a wearable ring device configured to be worn around a finger of the one or more arms of the user.

17. The system of claim 1, wherein the one or more sensors comprise one or more accelerometers, one or more gyroscopes, or both.

18. The system of claim 1, wherein the one more processors are further configured to:
determine one or more physical activities engaged in by the user during the second time interval based at least in part on the baseline motion data and the additional motion data.

19. A method for predicting illness onset, comprising:
acquiring, using one or more sensors of a wearable device, baseline motion data associated with one or more arms of a user, the baseline motion data acquired throughout a first time interval;
acquiring, using the one or more sensors of the wearable device, additional motion data associated with the one or more arms of the user, the additional motion data acquired via the one or more sensors of the wearable device throughout a second time interval that is subsequent to the first time interval;
inputting the baseline motion data and the additional motion data into one or more machine learning models, the one or more machine learning models trained to predict illness onset based at least in part on a plurality of features associated with movement of the one or more arms of the user within the additional motion data relative to the baseline motion data, the plurality of features comprising a first feature associated with a change in a hand swing movement range of the one or more arms, a second feature associated with a change in a shoulder angle range of the one or more arms, or both;
generating, using the one or more machine learning models, an illness prediction metric based at least in part on the plurality of features within the additional motion data, the illness prediction metric associated with a relative likelihood that the user is experiencing or will experience one or more illnesses; and
transmitting, to the wearable device, a user device, or both, an instruction configured to cause a graphical user interface (GUI) to display information associated with the illness prediction metric.

20. The method of claim 19, further comprising:
identifying, using the one or more machine learning models, one or more motion frequencies associated with one or more time intervals of the additional motion data associated with the one or more arms of the user; and
classifying, using the one or more machine learning models, the one or more time intervals of the additional motion data as being associated with a gait of the user or a tremor based at least in part on the one or more motion frequencies, wherein the plurality of features of the additional motion data further comprise the one or more motion frequencies associated with the one or more arms of the user.
